(19) United States
(12) Patent Application Publication
(10) Pub. No.: US 2004/0191897 A1
(43) Pub. Date: Sep. 30, 2004

(54) Apparatus and method for harvesting bone marrow

(75) Inventor: George F. Muschler, Cleveland Heights, OH (US)

Correspondence Address: TAROLLI, SUNDHEIM, COVELL & TUMMINO L.L.P., SUITE1111, 526 SUPERIOR AVENUE, CLEVELAND, OH, 44114-1400, US

(73) Assignee: The Cleveland Clinic Foundation

(21) Appl. No.: 10/813,986

(22) Filed: Mar. 31, 2004

Related U.S. Application Data
(60) Provisional application No. 60/459,199, filed on Mar. 31, 2003.

(51) Int. Cl. C12N 5/00

(52) U.S. Cl. 435/325

(57) ABSTRACT

A minimally invasive apparatus and method for harvesting bone marrow cells, blood, and bone fragments includes a rigid cannula having a proximal end and a distal end with an opening. The distal end includes a cutting tip that is movable axially and radially to cut and disrupt bone tissue while preserving necessary viability among harvested marrow cells. The cannula further includes an inner surface defining an internal passage that extends from the opening toward the proximal end. Suction is applied to the passage to draw disrupted bone marrow cells, blood, and bone fragments into the internal passage for collection. The apparatus may include a rotatable shaft disposed co-axially within the internal passage. The shaft has a distal end with a cutting bit for further cutting and disrupting of bone tissue.

### RELATED APPLICATION

This application claims priority from U.S. provisional patent application Ser. No. 60/459,199, filed on Mar. 31, 2003, the subject matter of which is incorporated herein by reference.

### FIELD OF THE INVENTION

The present invention relates to an apparatus and method for harvesting bone marrow and, in particular, is directed to a minimally invasive apparatus and method for harvesting bone marrow cells, blood, and bone fragments.

### BACKGROUND OF THE INVENTION

Each year, approximately one million bone grafting procedures are performed in the U.S. to treat acute fractures, fracture non-unions, bone defects, and to achieve therapeutic arthrodesis. Autogenous cancellous bone is currently the most effective graft material, and is used in approximately fifty percent of these procedures. However, the harvest of autogenous bone is associated with significant morbidity such as, surgical scars, blood loss, pain, prolonged surgical time and rehabilitation, increased exposure to blood products, and infection risk. Alternative procedures for bone harvest using osteotomes, curettes, reamers, and coring devices are all associated with these complications. The incidence of major complications associated with autogenous bone harvest has been estimated at eight percent. Many authors have reported on the complications associated with current methods of bone graft harvest.

This high cost of autograft harvest has resulted in graft interest and development of alternative methods and materials for bone grafting in an effort to avoid these complications, while still providing suitable biologic function. Several acellular alternatives have been developed in the past decade, including the use of allograft bone and some synthetic materials. However, none have been shown to be definitively better than autogenous cancellous bone, particularly in surgical spinal fusion and in treatment of delayed union and non-union of fractures in compromised tissue beds.

The effectiveness of any successful bone graft material is generally attributed to one or more of three core properties: osteoconduction, osteoinduction, and osteogenic cells. Autogenous cancellous bone, the current “gold standard”, has all three of the core properties. In addition, the graft site must provide a viable tissue surface from which revascularization can take place and a mechanical environment suitable to promote or allow bone differentiation.

Osteoconduction can be defined as a scaffold function provided by a graft material that facilitates the attachment and migration of cells involved in the healing process, and thereby the distribution of a bone healing response throughout the grafted volume. Cells of importance may include osteogenic cells, vascular endothelial cells, and others. Scaffold surface and architecture both contribute to osteoconduction.

Osteoinduction, in the broadest definition, refers to stimuli which promote osteoblastic progenitors to become activated, migrate, proliferate, and differentiate into bone forming cells. The prototypical stimuli are the family of bone morphogenetic proteins (BMPs). Both osteogenic and non-osteogenic cells, including endothelial cells, may elaborate inductive factors.

Osteogenic cells are progenitors that are capable of osteoblastic differentiation, found in the local bone or periosteum, or perivascular cells (e.g. vascular pericytes). They may also be transplanted with autogenous marrow. It is this last element, the presence of a high concentration of osteogenic cells and bone marrow derived cells, which separates autogenous cancellous bone from all other osteoconductive and oesteoinductive materials.

A practical source of exogenous osteoblastic progenitors is bone marrow harvested by aspiration. The value of bone marrow as a cell source for bone grafting has been supported by many studies, mainly in rodents, using marrow harvested by irrigation from long bones or open harvesting. Other studies have evaluated carrier matrices for the delivery of marrow cells. Much less evaluation of marrow grafting has been done on larger animals. In canine ulnar defects, it has been found that adding marrow to a collagen/ceramic composite material improved the mechanical result. It has also been found that the injection of heparinized marrow with demineralized bone powder improved the radiographic and mechanical result in canine tibial non-unions. Others have found that canine bone marrow is much less osteogenic than rabbit marrow when transplanted in diffusion chambers.

Several uncontrolled clinical series also imply that aspirated bone marrow has value. One report discusses the successful treatment of 18 of 20 tibial non-unions using bone marrow as the sole graft. Another report discusses healing in five to eight non-unions in oncology patients following marrow injections. It has also been shown that osteogenesis in a diffusion chamber was increased by adding a concentration of low density nucleated cells (<1.075 g/ml) from whole rabbit bone marrow, suggesting that the efficacy of bone marrow might be improved by intraoperative processing. Recognizing the potential biologic value and low risk, many surgeons have begun to use bone marrow as an adjuvant when allografting. However, published work has demonstrated that the concentration of osteogenic cells in bone marrow aspirates is diluted significantly (20-40 fold) below the concentration of osteoblastic cells present in cancellous bone. This dilution may limit the efficacy of bone marrow grafts. While dilution can be limited by reducing the aspiration volume to 2 cc or less, the surgeon is left with the challenge of performing many individual aspirations as well as the possible need to further re-concentrate these cells in order to optimize graft function.

The efficiency of harvest using existing needle devices is limited because these devices are designed for unidirectional (coaxial) function and harvest of bone marrow from a stationary needle site. As a result, each aspiration liberates bone marrow cells from only a very small area at the tip of the needle and minimal bone tissue is disrupted around the needle tip. This has advantages for biopsy procedures, but has significant disadvantages for bulk harvest of bone marrow cells.

Tools with cutting tips, such as gauges, curettes, trephines, and osteotomes have been designed as means for harvesting bone tissue. However, these tools are limited in that each requires repeated passage of the tool into and out of the bone tissue in order to accomplish bone harvest. In the case of gauges and osteotomes, these tools are only able to disrupt bone but have no means of retaining the disrupted tissue and therefore cannot deliver the disrupted tissue into the hands of the surgeon. As a result, these tools must be used as part of open procedures and alternative means of collecting the bone tissue, such as manual forceps or grabbing instruments, must be used.

Curettes and trephines can be used more percutaneously and can both disrupt and deliver bone tissue. However, both of these instruments are limited in efficiency because they can only be used to harvest small amounts of bone at one time and often must be inserted and removed many times in order to retrieve clinically useful amounts of tissue. Furthermore, in the case of both curettes and trephine instruments, these tools preferentially retain bone matrix in contrast to bone marrow. The softer and more fluid bone marrow contents of bone are frequently pressed out of the sample and lost into the wound.

One existing device has been designed and marketed for use in bulk harvest of cancellous bone and bone marrow. This device, which is embodied in U.S. Pat. No. 6,325,806, utilizes a long drill bit to disrupt bone and marrow contents. This drill functions at high speed (600 rpm) and collects bone and marrow contents by allowing the contents to be pulled up the through a sheath tightly surrounding the drill as a result of the suction created by spiral ramping of material along the threads of the drill. This provides an effective means of pulling bulk chunks of bone matrix into a chamber along with clotted blood and bone marrow. However, this device exposes the cells that are harvested to very high sheer stress, resulting in significant cell trauma and debris. In addition, this apparatus does not provide means for control of suction, irrigation, or anticoagulation. As a result, it is unsuitable as a tool for harvest of bone marrow cells that are in a state of health, viability, or physical form suitable for use in bone marrow transplantation and tissue engineering applications. Furthermore, this strategy also selectively harvests the bone matrix component of bone tissue and undesirably allows bone marrow to be pressed out of the bone tissue sample and become lost in the wound tissue or bleeding that escapes from collection at the drill tip.

Despite the development of matrix materials and growth factors that may enhance the bone healing process, it is believed that optimal performance of these materials will never be achieved without also delivering an optimal concentration of osteogenic cells in an appropriate environment. The bone marrow space within cancellous bone remains the most abundant and accessible source of these critical cells. Harvesting of cells through bone marrow aspiration and then concentrating the cells by various means can be used to improve the outcome of bone grafting. However, the clinical utility and value of bone marrow derived cells and the available processes for concentration of marrow cells would be greatly enhanced if an apparatus and associated methods were available to harvest large volumes of bone marrow safely and with minimally invasive techniques. The desired apparatus and methods would increase the number of cells that are available for bone marrow transplantation, bone grafting, and/or other tissue engineering applications. A wide range of tissue healing applications and both autograft and allograft cell therapy strategies would be enabled by such a desired apparatus and methods, due to the exceptionally broad range of biological potential that has been shown in bone marrow derived cells, including differentiation into blood cells, bone cartilage, tendon, ligament, skeletal muscle, fat, vascular tissue, endothelium, cardiac muscle, smooth muscle, nerve, liver, brain, and gut tissues. Further, the desired apparatus and methods would decrease the morbidity of bone marrow harvest and time required for marrow harvest, as well as preserve a high level of viability of the cells harvested.

### SUMMARY OF THE INVENTION

The present invention is a minimally invasive apparatus for harvesting bone marrow cells, blood, and bone fragments. The apparatus comprises a rigid cannula having a proximal end and a distal end with an opening. The distal end includes a cutting tip that is movable axially and radially to cut and disrupt bone tissue while preserving necessary viability among harvested marrow cells. The cannula further includes an inner surface defining an internal passage that extends from the opening toward the proximal end. Means are provided for applying suction to the passage in the cannula for drawing bone marrow cells, blood, and bone fragments disrupted from the bone tissue by the cutting tip into the passage for collection.

In accordance with one feature of the present invention, the apparatus further comprises a rotatable shaft disposed co-axially within the passage in the cannula. The shaft has a distal end with a cutting bit for disrupting bone tissue while preserving necessary viability among harvested marrow cells. Means are provided for rotating the shaft and the cutting bit. The apparatus can include control means for controlling the means for rotating the shaft.

In accordance with another feature of the present invention, the apparatus includes control means for controlling the means for applying suction.

In accordance with yet another feature of the present invention, the apparatus includes means for controllably supplying irrigation fluid to the distal end of the shaft.

In accordance with still another feature of the present invention, the apparatus includes means for controllably injecting an anticoagulant fluid into the harvested bone marrow cells, blood, and bone fragments during collection.

In accordance with still another feature of the present invention, the cannula further includes a plurality of nozzles adjacent the distal end for introducing anticoagulant fluid into harvested bone marrow cells, blood, and bone fragments immediately following their harvest.

In accordance with yet another feature of the present invention, the apparatus includes a collection chamber in fluid communication with the passage for receiving and collecting the harvested bone marrow cells, blood, and bone fragments.

In accordance with yet another feature of the present invention, the cannula includes a plurality of circumferentially spaced radially extending apertures through which bone marrow cells, blood, and bone fragments disrupted from the bone tissue are aspirated by the means for applying suction.

In accordance with still another feature of the present invention, the apparatus includes a sheath disposed coaxially about a portion of the cannula for providing and maintaining a single puncture site.

In accordance with still another feature of the present invention, the opening at the distal end of the cannula extends in the radial direction through the inner surface of the cannula.

In accordance with still another feature of the present invention, the distal end of the cannula includes a plurality of fluid nozzles adjacent the radial opening for introducing an anticoagulant fluid into harvested bone marrow cells, blood, and bone fragments.

In accordance with still another feature of the present invention, the cannula includes a radially extending wall that closes off a portion of the internal passage at the distal end of the cannula. The end wall has an axially extending passage through which the shaft projects.

The present invention also provides a minimally invasive method for harvesting bone marrow cells, blood, and bone fragments. According to the inventive method, a rigid cannula having a proximal end and a distal end with an opening is provided. The distal end includes a cutting tip cutting and for disrupting bone tissue while preserving necessary viability among harvested marrow cells. The cannula further includes an inner surface defining an internal passage that extends from the opening toward the proximal end. The distal end of the cannula is inserted through a puncture site, through the cortex of a bone, and into the intramedullary canal of the bone. Suction is applied to the passage which draws bone marrow cells, blood, and bone fragments disrupted from the cancellous bone into the passage for collection. The distal end of the cannula is then manually moved in both axial and radial directions within the intramedullary canal to cut and disrupt the bone tissue. The distal end of the cannula is also manually moved to different locations in the cancellous bone to disrupt additional bone tissue while remaining in the same puncture site.

In accordance with a feature of the present invention, the method further comprises providing a rotatable shaft disposed co-axially within the passage in the cannula. The shaft has distal end with a cutting bit that projects through the opening in the cannula. The shaft is rotated to disrupt bone tissue in the intramedullary canal while preserving necessary viability among harvested marrow cells.

In accordance with another feature of the present invention, the method further comprises supplying irrigation fluid to the distal end of the shaft to minimize thermal or mechanical trauma to the harvested cells and to help carry the harvested bone marrow cells, blood, and bone fragments into the passage.

In accordance with another feature of the present invention, the method further comprises supplying an anticoagulant fluid into the harvested bone marrow cells, blood, and bone fragments to inhibit clot formation.

In accordance with another feature of the present invention, the method further comprises the step of oscillating the supply of irrigation fluid and the supply of anticoagulant fluid.

In accordance with another feature of the present invention, the method further comprises the step of supplying the anticoagulant fluid at a location adjacent the distal end of the cannula.

In accordance with yet another feature of the present invention, the method further comprises providing a collection reservoir for collecting the harvested bone marrow cells, blood, and bone fragments. The passage in the cannula is fluidly connected with the collection reservoir.

In accordance with still another feature of the present invention, the method further comprises providing a sheath that is disposed co-axially about a portion of the cannula. The sheath is inserted percutaneously to the cortex of the bone so that the sheath provides and maintains a single percutaneous puncture site for harvesting bone marrow cells, blood, and bone fragments.

In accordance with another feature of the present invention, the method further comprises the step of aspirating bone marrow cells, blood, and bone fragments disrupted from the bone tissue through a plurality of circumferentially spaced radially oriented apertures at the distal end of the cannula.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic anterior view of a pelvis and illustrates an apparatus for harvesting bone marrow cells, blood, and bone fragments in accordance with present invention;

FIG. 2 is an enlarged view of a portion of FIG. 1 and illustrates a feature of the present invention that is used to gain access to the pelvis;

FIG. 3 is a schematic side view of the apparatus of FIG. 1;

FIG. 4 is a view similar to FIG. 3 showing the apparatus inserted into the ilium;

FIG. 5A is a schematic oblique lateral view of the apparatus of FIG. 3 inserted into the ilium in an initial stage in the harvesting process;

FIG. 5B is a view similar to FIG. 5A illustrating an intermediate stage in the harvesting process;

FIG. 5C is a view similar to FIG. 5B illustrating a subsequent intermediate stage in the harvesting process;

FIG. 5D is a view similar to FIG. 5C illustrating a final stage in the harvesting process;

FIG. 5E is a lateral view of a left coxal bone illustrating an optional additional apparatus inserted at a second location in the ilium and at the final stage of an additional harvesting process;

FIG. 5F is a sectional view taken along line 5F-5F in FIG. 5E;

FIGS. 6-9 are perspective views of alternate configurations for the distal end portion of the apparatus of FIG. 3;

FIG. 10 is a view similar to FIG. 4 illustrating an apparatus constructed in accordance with a second embodiment of the present invention;

FIG. 11 is a view similar to FIG. 4 illustrating an apparatus constructed in accordance with a third embodiment of the present invention;

FIG. 12 is a view similar to FIG. 3 illustrating an apparatus constructed in accordance with a fourth embodiment of the present invention;

FIG. 13 is an enlarged view of illustrating an alternate configuration for an end portion of the apparatus of FIG. 12;

FIG. 14 is a side view illustrating another alternate configuration for the end portion of the apparatus of FIG. 12;

FIG. 15 is a view similar to FIG. 3 illustrating an apparatus constructed in accordance with a fifth embodiment of the present invention;

FIG. 16 is a view similar to FIG. 3 illustrating an apparatus constructed in accordance with a sixth embodiment of the present invention;

FIG. 17 is a view illustrating an apparatus constructed in accordance with a seventh embodiment of the present invention;

FIG. 18 is a view similar to FIG. 17 with parts omitted for clarity;

FIG. 19 is a perspective view illustrating an apparatus constructed in accordance with an eighth embodiment of the present invention;

FIG. 20 is a view similar to FIG. 19 illustrating an alternate construction;

FIG. 21 is a view similar to FIG. 19 illustrating an apparatus constructed in accordance with a ninth embodiment of the present invention; and

FIG. 22 is a view similar to FIG. 21 illustrating an apparatus constructed in accordance with a tenth embodiment of the present invention.

### DESCRIPTION OF EMBODIMENTS

The present invention relates to an apparatus and method for harvesting bone marrow and, in particular, is directed to a minimally invasive apparatus and method for harvesting bone marrow cells, blood, and bone fragments. As representative of the present invention, FIG. 1 illustrates a human pelvis 10 which includes the ilium 12, the ischium 14, the pubis 16, and the sacrum 18. FIG. 1 also illustrates an apparatus 20 for harvesting bone marrow cells, blood, and bone fragments in accordance with a first embodiment of the present invention.

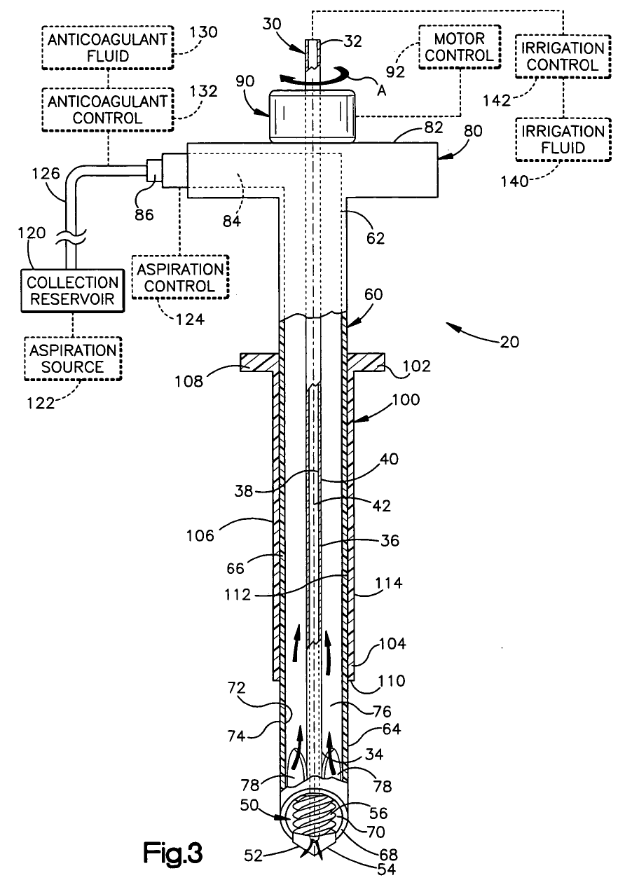

As best seen in FIG. 3, the apparatus 20 comprises a rotatable shaft 30, a cannula 60, and a sheath 100. The shaft 30 is a rigid tubular member with an outer diameter of 1-3mm. The shaft 30 has a proximal end 32, a distal end 34, and a main body portion 36 extending between the two ends. The main body portion 36 includes cylindrical inner and outer surfaces 38 and 40, respectively. The inner surface 38 defines a lumen 42 that extends from the proximal end 32 to the distal end 34 of the shaft 30.

A cutting bit 50 for disrupting bone marrow cells, blood, and bone fragments from the bone tissue, while preserving necessary viability among harvested marrow cells, is attached to the distal end 34 of the shaft 30. The cutting bit 50 may have a number of different configurations, as is discussed further below, and has a maximum outer diameter of 3-8mm. In the embodiment of FIG. 3, the cutting bit 50 includes an oppositely disposed pair of converging cutting edges 52 and 54 and a set of helical threads 56. The helical threads 56 resemble a drill bit and extend proximally away from the cutting edges 52 and 54. The lumen 42 in the shaft 30 extends into the cutting bit 50 to one or more apertures 58 at the terminal end of the helical threads.

The cannula 60 is also a rigid tubular member and is disposed coaxially about the main body portion 36 of the shaft 30. The cannula 60 has a proximal end 62, a distal end 64, and a main body portion 66 that is 10-40 cm in length and extends between the ends. The distal end 64 includes an end surface 68 that defines an opening 70 through which the cutting bit 50 projects. The end surface 68 may be machined to a sharp edge for use in scraping and cutting bone tissue in conjunction with the cutting bit 50 or when the cutting bit is withdrawn or retracted into the cannula 60.

The main body portion 66 of the cannula 60 includes cylindrical inner and outer surfaces 72 and 74, respectively. The inner surface 72 of the cannula defines an internal passage 76 with a diameter of 3-7 mm. Adjacent the distal end 64, the main body portion 66 of the cannula 60 includes a plurality of apertures 78 that are spaced circumferentially apart and that extend between the inner and outer surfaces 72 and 74. The apertures 78 illustrated in FIG. 3 have an oval shape, but could alternatively have another suitable shape.

The proximal end 62 of the cannula 60 includes a housing 80. The housing 80 can be of any suitable shape, such as cylindrical. The passage 76 in the cannula 60 extends into the housing 80 as may be seen in FIG. 3. The shaft 30 projects out of the housing 80 through an upper surface 82 of the housing. It is contemplated that a bearing (not shown) could be located near the upper surface 82 of the housing 80 to support the rotatable shaft 30. The housing 80 includes a radially extending channel 84 that intersects the passage 76 in the cannula 60. The channel 84 fluidly connects the passage 76 to an outlet coupling 86 on the exterior of the housing 80.

In accordance with one embodiment of the present invention, a motor 90 is attached to the upper surface 82 of the housing 80. The shaft 30 extends through the motor 90 as shown in FIG. 3, but need not do so. The motor 90 is operable to rotate the shaft, as indicated by arrow A. The motor 90 may be electric, pneumatic, hydraulic, or energized by any other suitable means. The motor 90 should have variable speed capability and be able to operate at 60-600 RPM. Energization and speed of the motor 90 is controlled by a motor control 92. The motor control 92 is manually operated by a surgeon and may be of any suitable construction, including but not limited to a knob, a trigger, or a foot pedal. Alternatively, it is contemplated that the shaft 30 could instead be rotated with the assistance of a mechanical device or mechanism (not shown). In such a circumstance, the motor 90 would thus be omitted from the apparatus 20.

The sheath 100 is another rigid tubular member and is disposed coaxially about a portion of the main body portion 66 of the cannula 60. The sheath 100 has a proximal end 102, a distal end 104, and a main body portion 106 extending between the two ends. The distal end 104 includes an annular flange 108, while the proximal end 102 terminates at an annular surface 110. The main body portion 106 is 5-20 cm in length and includes cylindrical inner and outer surfaces 112 and 114, respectively. The inner surface 112 defines a lumen (not numbered) that extends from the proximal end 102 to the distal end 104 of the sheath 100. The inner surface 112 is dimensioned to fit tightly over the outer surface 74 of the cannula 60 but allow relative movement between the cannula and the sheath 100. The diameter of the outer surface 114 of the sheath is 4-10 mm.

The apparatus 20 further includes a collection reservoir 120, an aspiration source 122 that creates a negative pressure (or vacuum) through the collection reservoir, and a controller 124 for controlling the aspiration. The collection reservoir 120 is fluidly connected via a suction line 126 to the outlet coupling 86 on the housing 80. The collection reservoir 120, the details of which are not shown, can have one or more levels of filtering and multiple internal chambers for separating blood from harvested bone marrow cells, blood, and bone fragments.

The aspiration source 122 can be any suitable means for pulling a vacuum, such as the typical surgical aspiration line found in most operating rooms or a stand-alone vacuum pump dedicated for use with the apparatus 20. The aspiration controller 122 is manually operated by the surgeon and may be of any suitable construction, including but not limited to a knob, a trigger, or a foot pedal. As shown schematically in FIG. 3, the aspiration control 124 may be conveniently located on or at the housing of the cannula. The aspiration control 124 should be able to provide either continuous or variable aspiration of the passage 76 in the cannula 60. The aspiration control 124 could also be able to provide oscillatory aspiration.

In accordance with one feature of the present invention, the apparatus 20 includes a pressurized source 130 of an anticoagulant fluid, such as heparinized saline, and a control 132 for controlling the flow of anticoagulant fluid. The source 130 of anticoagulant fluid and the associated control 132, both of which are shown only schematically in FIG. 3, are fluidly connected to the suction line 126 so that the anticoagulant fluid can be injected into the harvested bone tissue as it is drawn through the suction line and into the collection reservoir 120. The anticoagulant control 132 should be manually operable by the surgeon and may be of any suitable construction, including but not limited to a knob, a trigger, or a foot pedal. Further, the anticoagulant control 132 should be able to provide for either continuous or variable flow of anticoagulant fluid into the suction line 126. The anticoagulant control 132 could also provide be able to provide oscillatory flow of anticoagulant fluid.

In accordance with another feature of the invention, the apparatus 20 further includes a pressurized source 140 of an irrigation fluid, such as saline, and a control 142 for controlling the flow of irrigation fluid. The source 140 of irrigation fluid and the associated control 142, both of which are shown only schematically in FIG. 3, are fluidly connected to the proximal end 32 of the shaft 30 that projects out of the motor 90. A rotating seal (not shown) may be used to fluidly connect the irrigation fluid source 140 to the rotatable shaft 30. Irrigation fluid can then be supplied, via the lumen 42 through the shaft 30, to the cutting bit 50 to help cool the cutting bit and, if necessary, clear tissue (bone or other) lodged in the cutting bit. The irrigation fluid may also function as a carrier medium to aid in the aspiration of the disrupted bone tissue. The irrigation fluid control 142 should be manually operable by the surgeon and may be of any suitable construction, including but not limited to a knob, a trigger, or a foot pedal. Further, the irrigation fluid control 142 should be able to provide for either continuous or variable flow of irrigation fluid into the lumen 42 in the shaft 30. The irrigation control 142 could also be able to provide oscillatory flow of irrigation fluid.

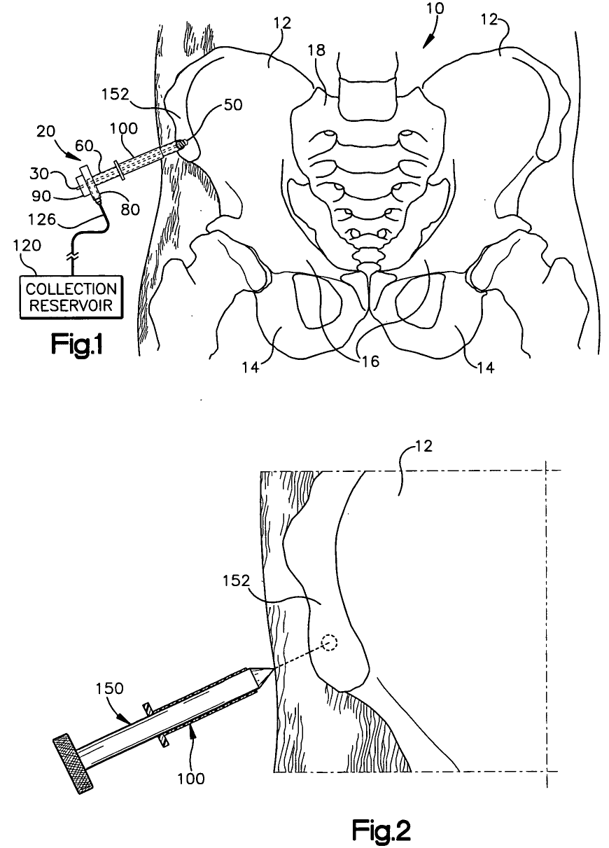

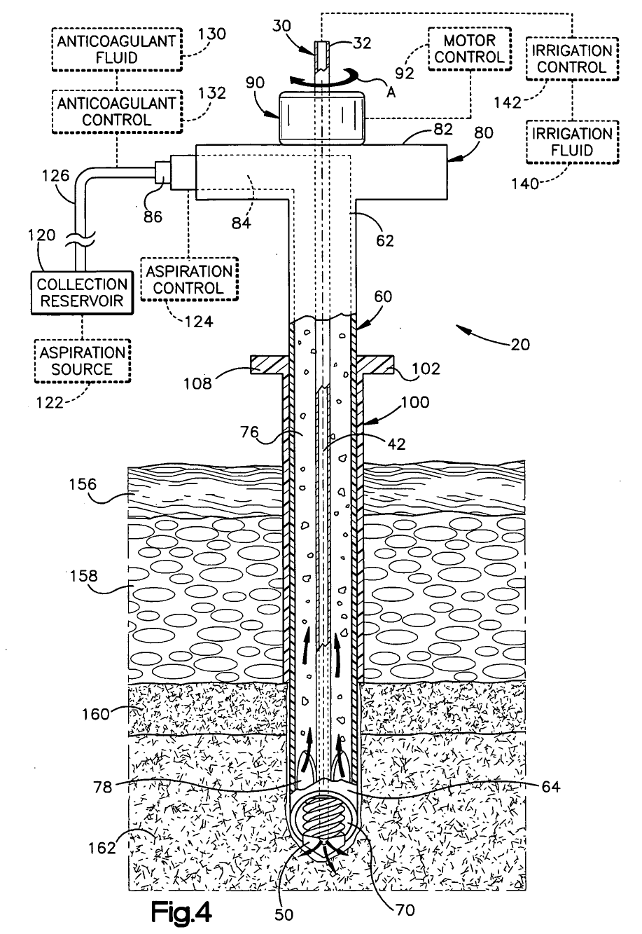

In order to use the apparatus 20 described above to harvest bone marrow cells, blood, and bone fragments from the ilium 12, access to the iliac crest must first be achieved. FIG. 2 illustrates an obturator 150 disposed within the sheath 100 for use in accessing the iliac crest 152. The obturator 150 is of known construction and includes a pointed tip 154 for penetrating through the skin layer(s) 156 (see FIG. 4) and the fatty tissue layer(s) 158 to the cortical bone 160 of the ilium 12. Once this penetration has been achieved, the obturator 150 is withdrawn from the sheath 100, which remains in a percutaneous position to form a percutaneous access passage for receiving the cannula 60 of the apparatus 20. As shown in FIG. 1, the cannula 60 and the shaft 30 can then be inserted into the lumen in the sheath 100 to begin cutting into the iliac crest 152. It is contemplated, but not required, that prior to the insertion of the shaft 30 with the cutting bit 50 into the sheath 100, a known drill-type device (not shown) may be inserted first and used to cut through the cortical bone 160 and into the intramedullary canal of the ilium 12.

As may be seen in FIG. 4, once the distal end 64 of the cannula 60 has been pushed through the cortical bone 160 into the intramedullary canal, the harvesting of bone marrow cells, blood, and bone fragments from the cancellous bone 162 in the intramedullary canal can begin. The shaft 30 and thus the cutting bit 50 are rotated as indicated by arrow A, causing bone marrow and bone fragments to be disrupted from the cancellous bone 162 while still preserving necessary viability among harvested marrow cells. In order to reduce the chance of thermal or mechanical trauma to the bone marrow cells being harvested, irrigation fluid may be dispensed through the lumen 42 and out through the cutting bit 50 to cool the cutting bit. The flow of irrigation fluid may also serve to clear any bone or other tissue that has become lodged in the cutting bit 50 or the distal end 64 of the cannula 60.

The disrupted bone marrow cells, blood, and bone fragments are then aspirated into the passage 76 in the cannula 60 through either the opening 70 at the distal end 64 of the cannula or through the apertures 78 adjacent the distal end. Disrupted bone marrow cells, blood, and bone fragments that find their way into the helical threads 56 on the cutting bit 50 are aided into the passage 76 by the rotation of the helical threads. Aspiration of the disrupted bone marrow cells, blood, and bone fragments can also be augmented by the introduction of irrigation fluid which is primarily used to cool the cutting bit 50, but which also functions as a carrier medium to wash disrupted bone marrow cells, blood, and bone fragments into the passage 76. During the aspiration process, should any bone or other tissue become lodged within the apertures 78 in the cannula 60, it may be possible to clear the material from the apertures by withdrawing the cannula into the sheath 100 so that the distal end 102 of the sheath 100 can shear off and release any such lodged material.

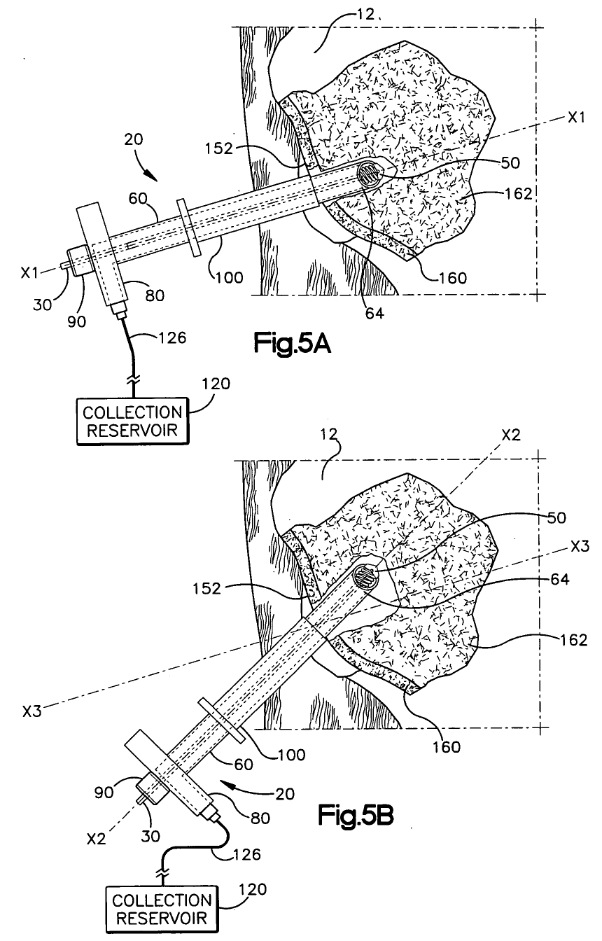

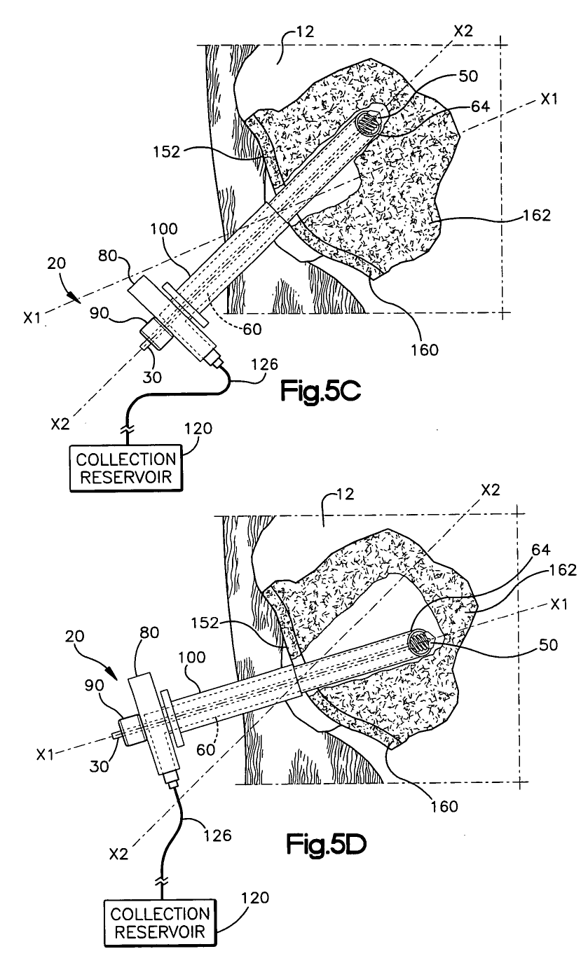

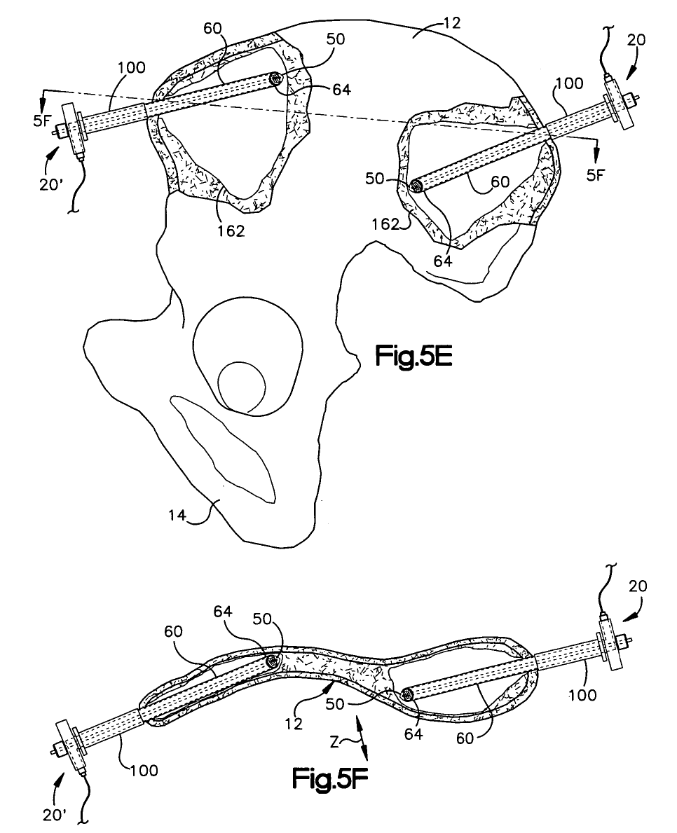

The disrupted bone marrow cells, blood, and bone fragments aspirated into the passage 76 in the cannula 60 are pulled through the passage and out of the cannula through the channel 84 and the outlet coupling 86. As the harvested bone marrow cells, blood, and bone fragments pass through the suction line 126 on their way to the collection reservoir 120, the anticoagulant fluid may be introduced into the suction line to inhibit clotting and thus help preserve the harvested material for its intended use. It should be noted FIGS. 5A-D illustrate how the apparatus 20 (with certain features omitted for clarity) is used to harvest a large volume of bone marrow cells, blood, and bone fragments from a single access site. In the initial stage of the harvesting process shown in FIG. 5A, the distal end 64 of the cannula 60 has been inserted into the cancellous bone 162 and moved along a first axis X1 to harvest a first portion of material (bone marrow cells, blood, and bone fragments). In addition to the material disruption by the cutting bit 50, the rigid structure of the cannula 60 allows the surgeon to also use the distal end 64 of the cannula 60 to scrape and disrupt the cancellous bone 162 for harvest.

Next, the surgeon moves the cannula 60 such that the distal end 64 deviates from the first axis X1 and moves toward a second axis X2 (FIG. 5B). During this angular movement, the rotating cutting bit 50 and the distal end 64 of the cannula 60 are used to continue excavating material within the intramedullary canal. By the time the cannula 60 has been moved far enough that it now lies on the second axis as is shown in FIG. 5B, a second portion of material has been harvested and a small pie-shaped void in the ilium 12 has been created. The rigid cannula 60 provides helpful tactile feedback to the surgeon so that the distal end 64 of the cannula does not leave the intramedullary canal.

As may be seen in FIG. 5C, the distal end 64 of the cannula 60 is then advanced further into the intramedullary canal along the second axis X2 to harvest a third portion of material. Finally, the cannula 60 is gradually moved from its position along the second axis X2 back toward the first axis X1. During this movement, the rotating cutting bit 50 and the distal end 64 of the cannula 60 continuously excavate material within the intramedullary canal. As shown in FIG. 5D, when the cannula 60 has been moved far enough so that it lies on a third axis X3, which may or may not be coincident with the first axis X1, a fourth portion of material has been harvested and a larger pie-shaped void in the intramedullary canal has been created.

Throughout the process described above, based on factors such as the bone characteristics and desired harvest (type and quantity), the surgeon may vary following parameters: the rotational speed of the cutting bit 50 using the motor control 92, the aspiration pressure using the aspiration control 124, the flow rate of irrigation fluid using the irrigation control 142, and the flow rate of anticoagulant fluid using the anticoagulant control 132.

It should be understood that the pattern and order in which the material is harvested from the intramedullary canal that is described above is exemplary in nature and is not intended to be dispositive in any way. It is envisioned that a wide variety of variations in depth of penetration and angular movement of the distal end 64 of the cannula 60 within the intramedullary canal could be employed, particularly if variations in bone characteristics exist.

Using the aforementioned process and the apparatus 20, large volumes of bone can be safely harvested and in a minimally invasive fashion. Due to the minimally invasive nature of the apparatus 20 and associated method for harvesting bone marrow cells, the present invention increases the number of harvested cells that are available for bone marrow transplantation, bone grafting, and/or tissue engineering. Further, the minimally invasive apparatus 20 and method described above decreases the morbidity of bone marrow harvest and the time required for marrow harvest.

FIGS. 5E and 5F further illustrate how the apparatus 20 is used to harvest large volumes of bone marrow cells, blood, and bone fragments. In FIGS. 5E and 5F, a second site on the opposite side of the ilium 12 is accessed in addition to the site in the iliac crest 152. A second identically constructed apparatus 20′ is used to harvest bone marrow cells, blood, and bone fragments from the intramedullary canal in the opposite side of the ilium 12 in the same basic manner as described above. Alternatively and perhaps more likely, a single apparatus 20 could be used in both locations in consecutive procedures. As may be seen in FIG. 5F, the apparatus 20 can also be moved by the surgeon in the transverse plane, indicated by arrow Z, to increase the volume of bone marrow cells, blood, and bone fragments harvested from the intramedullary canal. Again, the rigid cannula 60 provides helpful tactile feedback to the surgeon so that the distal end 64 of the cannula stays within the intramedullary canal.

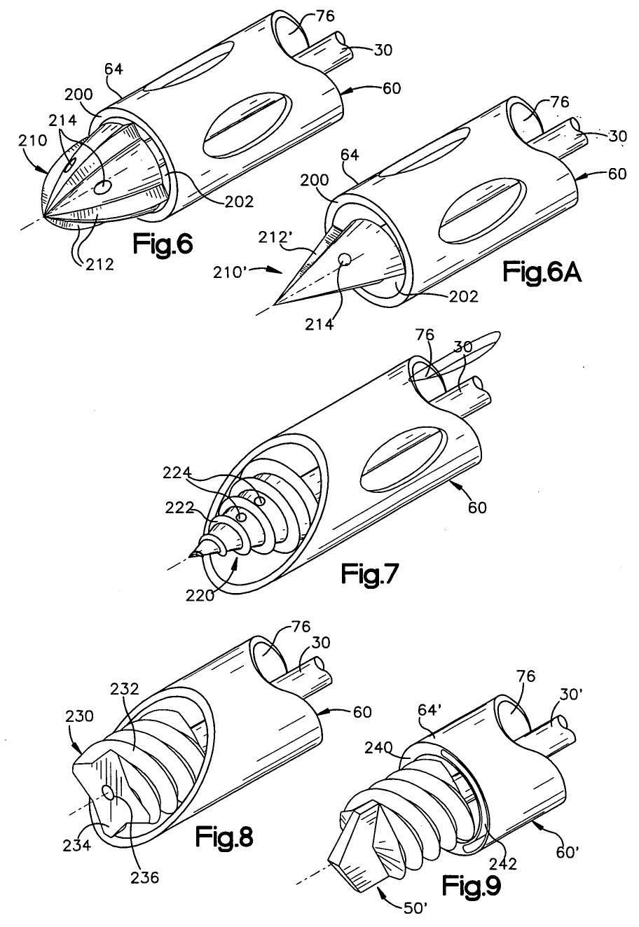

FIGS. 6-9 illustrate a number of alternate configurations for the distal end 64 of the cannula 60 and the cutting bit 50 on the shaft 30. In FIGS. 6-9, reference numbers that are the same as reference numbers used in FIGS. 1-4 identify parts and features that are the same as FIGS. 1-4.

In the alternate configuration of FIG. 6, the distal end 64 of the cannula 60 has an annular end surface 200 that surrounds an opening 202 into the passage 76. The shaft 30 includes a cutting bit 210 that is conical in shape and has a plurality of axially extending blades 212. Apertures 214 are located in the cutting bit 210 allow for irrigation. A variation of the cutting bit 210 of FIG. 6 is illustrated in FIG. 6A as cutting bit 210′ which has a pair of oppositely disposed axially extending blades 212′. In FIG. 7, the shaft 30 has a conical cutting bit 220 with helical threads 222. Apertures 224 are located in the cutting bit 220 allow for irrigation. In FIG. 8, the shaft 30 includes a generally cylindrical cutting bit 230 with helical threads 232 and a blunt tip 234. An opening 236 in the blunt tip 234 allows for irrigation.

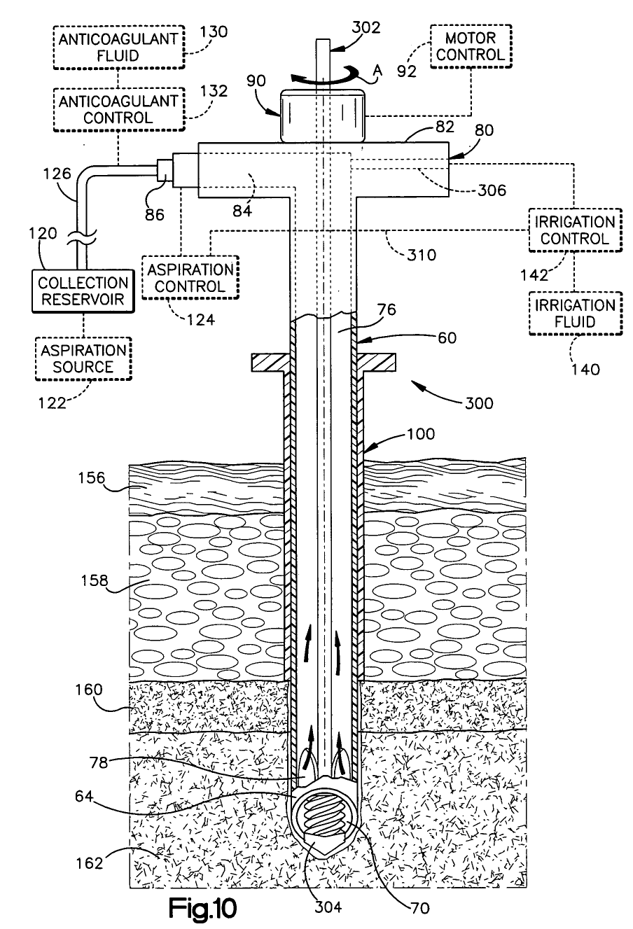

In the alternate configuration of FIG. 9, reference numbers marked with a prime (′) indicate parts with slightly different features from the first embodiment. In FIG. 9, the shaft 30′ is not hollow and the cutting bit 50′ does not include apertures for irrigation fluid flow. Rather, the distal end 64′ of the cannula 60′ has an annular end surface 240 that includes an opening 242 from an arcuate-shaped lumen (not numbered) in the side wall of the cannula. The lumen extends through the main body portion 66 of the cannula 60′ and is fluidly connected, in a manner not shown, to the irrigation fluid source 140.

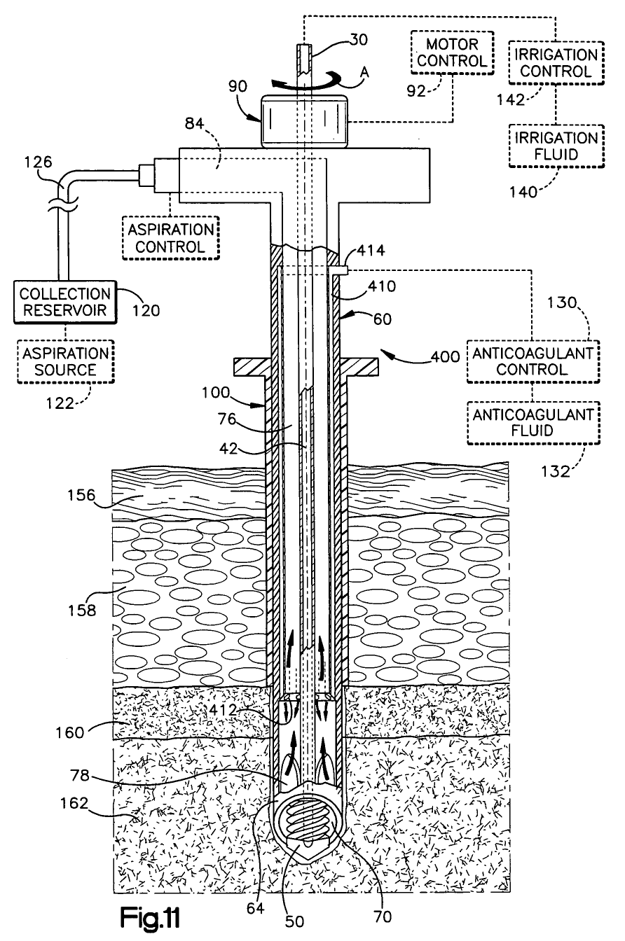

FIG. 10 illustrates an apparatus 300 constructed in accordance with a second embodiment of the present invention. In FIG. 10, reference numbers that are the same as reference numbers used in the first embodiment of FIGS. 1-4 identify parts and features that are the same as those in FIGS. 1-4.

According to the second embodiment, the apparatus 300 includes a shaft 302 and a cutting bit 304 that are solid rather than hollow. The irrigation fluid source 140 and irrigation control 142 are fluidly connected, in a manner not shown, to a radially extending channel 306 in the housing 80 of the cannula 60. The channel 306 leads into the passage 76 in the cannula 60.

The apparatus 300 is used to harvest bone marrow cells, blood, and bone fragments in much the same fashion as the apparatus 20 described above, except that irrigation and aspiration are done via the same fluid passage 76. Accordingly, the irrigation process and the aspiration process must be oscillated so as not to coincide with one another. This oscillation can be accomplished manually by the surgeon or automatically by operatively coupling the aspiration control 124 to the irrigation control 142 as indicated by the dotted line 310 in FIG. 10.

As with the first embodiment of FIGS. 1-4, the embodiment of FIG. 10 provides an apparatus 300 and method for harvesting large volumes of bone in a minimally invasive fashion. Due to the relatively atraumatic nature of the apparatus 300 and associated method for harvesting bone marrow cells described herein, the invention increases the number of harvested cells that are available for bone marrow transplantation, bone grafting, and/or tissue engineering. Further, the invention decreases the morbidity of bone marrow harvest and the time required for marrow harvest.

FIG. 11 illustrates an apparatus 400 constructed in accordance with a third embodiment of the present invention. In FIG. 11, reference numbers that are the same as reference numbers used in the first embodiment of FIGS. 1-4 identify parts and features that are the same as those in FIGS. 1-4.

According to the third embodiment, the cannula 60 of the apparatus 400 includes an annular lumen 410 in the side wall of the main body portion 66. Near the distal end 64 of the cannula 60, the lumen 410 terminates at a plurality of nozzles 412. The lumen 410 intersects an external port 414 near the proximal end 62 of the cannula 60. The anticoagulant fluid source 130 and associated control 132 are fluidly connected to the external port 414.

The apparatus of FIG. 11 is used to harvest bone marrow cells, blood, and bone fragments in much the same fashion as the apparatus 20 described above, except that the anticoagulant fluid can be introduced sooner into the harvested materials via the lumen 410 and the nozzles 412. The ability to introduce the anticoagulant fluid immediately after the harvested materials enter the passage 76 helps to preserve a high level of viability and to maintain the bone marrow cells harvested in suspension without formation of a fibrin clot.

As with the previous embodiments, the apparatus 400 and method allows for harvesting large volumes of bone in a minimally invasive fashion, increases the number of harvested cells that are available for bone marrow transplantation, bone grafting, and/or tissue engineering, and decreases the morbidity of bone marrow harvest and the time required for marrow harvest.

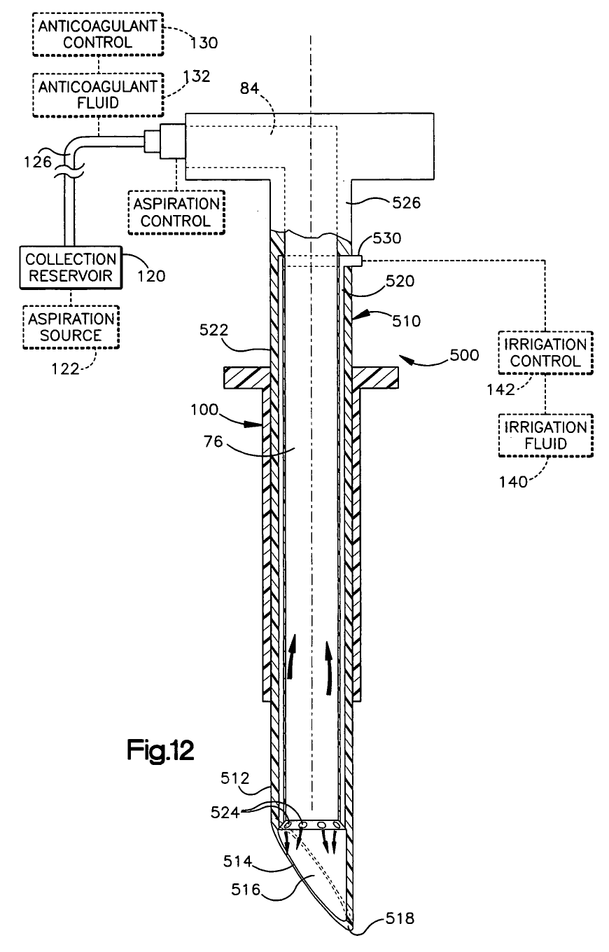

FIG. 12 illustrates an apparatus 500 constructed in accordance with a fourth embodiment of the present invention. In FIG. 12, reference numbers that are the same as reference numbers used in the first embodiment of FIGS. 1-4 identify parts and features that are the same as those in FIGS. 1-4.

According to the fourth embodiment, the apparatus 500 includes the sheath 100 and a rigid cannula 510 that is similar, but not identical to, the cannula 60 described above. In contrast to the earlier embodiments, the apparatus 500 does not include the shaft 50 with cutting bit 60, nor does it include the motor 90 and associated motor control 92.

The cannula 510 includes a distal end 512 that has an angled end surface 514 that defines an oblique opening 516 into the passage 76 in the cannula. As may be seen in FIG. 12, the angled end surface 514 has an elongated shape and terminates at a pointed cutting tip 518.

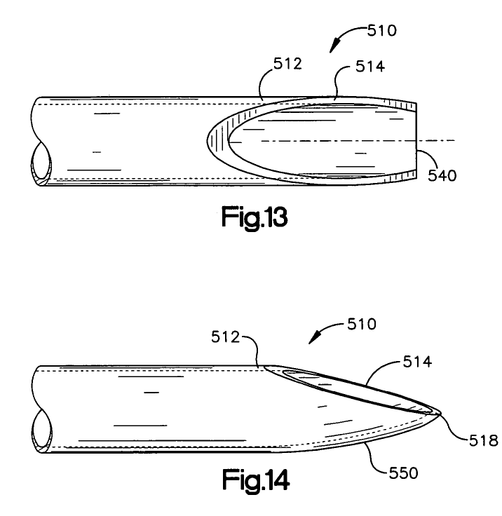

FIGS. 13 and 14 illustrate alternate configurations for the distal end 512 of the cannula 510. In FIG. 13, the angled end surface 514 terminates at a flattened cutting tip 540 that is designed to cut into bone like a chisel. In FIG. 14, the distal end 512 has a slight curvature 550 that resembles a curette.

The cannula 510 further includes an annular lumen 520 in the side wall of a main body portion 522 of the cannula. Near the distal end 512, the lumen 520 terminates at one or more nozzles 524. Near a proximal end 526 of the cannula 510, the lumen 520 intersects an external port 530. The irrigation fluid source 140 and associated control 142 are fluidly connected to the external port 530.

The apparatus 500 of FIG. 12 is used to harvest bone marrow cells, blood, and bone fragments in much the same manner as the apparatus 20 described above, except that the distal end 512 of the rigid cannula 510 and, in particular, the cutting tip 518 are the structure that is used to disrupt, scrape, and excavate cancellous bone from the intramedullary canal. The distal end 512′ of the cannula 510 is manually moved in both axial and radial directions to achieve this excavation. Irrigation fluid supplied to the cutting tip 518 via the lumen 520 and the nozzles 524 can assist in washing the disrupted bone tissue into the opening 516 and into the passage 76 in the cannula 510.

As with the previous embodiments, the apparatus 500 and method allows for harvesting large volumes of bone in a minimally invasive fashion, increases the number of harvested cells that are available for bone marrow transplantation, bone grafting, and/or tissue engineering, while decreasing the morbidity of bone marrow harvest and the time required for marrow harvest.

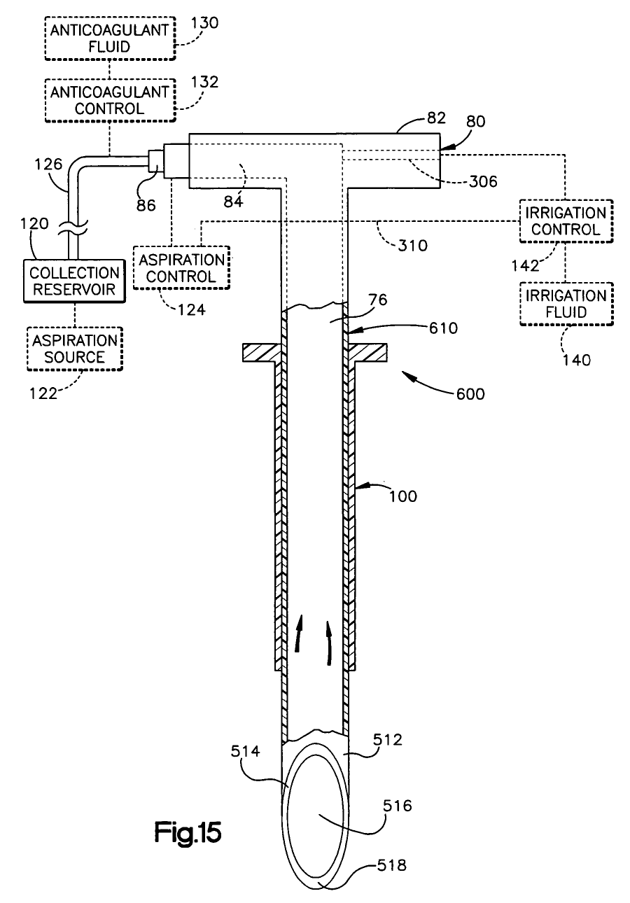

FIG. 15 illustrates an apparatus 600 constructed in accordance with a fifth embodiment of the present invention. In FIG. 15, reference numbers that are the same as reference numbers used in the previous embodiments identify parts and features that are the same as those in the previous embodiments.

According to the fifth embodiment, the apparatus 600 includes the sheath 100 and a rigid cannula 610 that is similar, but not identical to, the cannula 510 described above. As with the fourth embodiment of FIG. 12, the apparatus 600 of FIG. 15 does not include the shaft 50 with cutting bit 60, nor does it include the motor 90 and associated motor control 92.

The cannula 610 includes the distal end 512 and angled end surface 514 that defines the oblique opening 516 into the passage 76 in the cannula. The angled end surface 514 has an elongated shape and terminates at the pointed cutting tip 518.

The irrigation fluid source 140 and irrigation control 142 are fluidly connected, in a manner not shown, to the radially extending channel 306 in the housing 80 of the cannula 610. The channel 306 leads into the passage 76 in the cannula 610.

The apparatus 600 is used to harvest bone marrow cells, blood, and bone fragments in much the same fashion as the apparatus 500 described above, except that irrigation and aspiration are done via the same fluid passage 76. Accordingly, the irrigation process and the aspiration process must be oscillated so as not to coincide with one another. This oscillation can be accomplished manually by the surgeon or automatically by operatively coupling the aspiration control 124 to the irrigation control 142 as indicated by the dotted line 310 in FIG. 15.

As with the previous embodiments, the apparatus 600 and method allows for harvesting large volumes of bone in a minimally invasive fashion, increases the number of harvested cells that are available for bone marrow transplantation, bone grafting, and/or tissue engineering, while decreasing the morbidity of bone marrow harvest and the time required for marrow harvest.

FIG. 16 illustrates an apparatus 700 constructed in accordance with a sixth embodiment of the present invention. In FIG. 16, reference numbers that are the same as reference numbers used in the earlier embodiments identify parts and features that are the same as those in earlier embodiments.

According to the sixth embodiment, the rigid cannula 510 includes the distal end 512 with the oblique opening 516 into the passage 76 in the cannula. The cannula 510 further includes the annular lumen 520 in the side wall of a main body portion 522 of the cannula. Near the distal end 512, the lumen 520 terminates at one or more nozzles 524. Near a proximal end 526 of the cannula 510, the lumen 520 intersects an external port 530. The anticoagulant fluid source 130 and associated control 132 are fluidly connected to the external port 530.

The apparatus 700 further includes a tube 710 located coaxially within the cannula 510. The tube 710 has a proximal end 712 that projects from the housing 80 and which is fluidly connected to the irrigation fluid source 140 and associated control 142. A distal end 714 of the tube 710 is located near the opening 516 in the cannula. The distal end 714 is supported in the passage 76 by struts 720.

The apparatus of FIG. 16 is used to harvest bone marrow cells, blood, and bone fragments in much the same fashion as the apparatus 500 described above, except that irrigation fluid is supplied to the cutting tip 518 via the tube 710. As with the previous embodiments, the apparatus 700 and method allows for harvesting large volumes of bone in a minimally invasive fashion, increases the number of harvested cells that are available for bone marrow transplantation, bone grafting, and/or tissue engineering, while decreasing the morbidity of bone marrow harvest and the time required for marrow harvest.

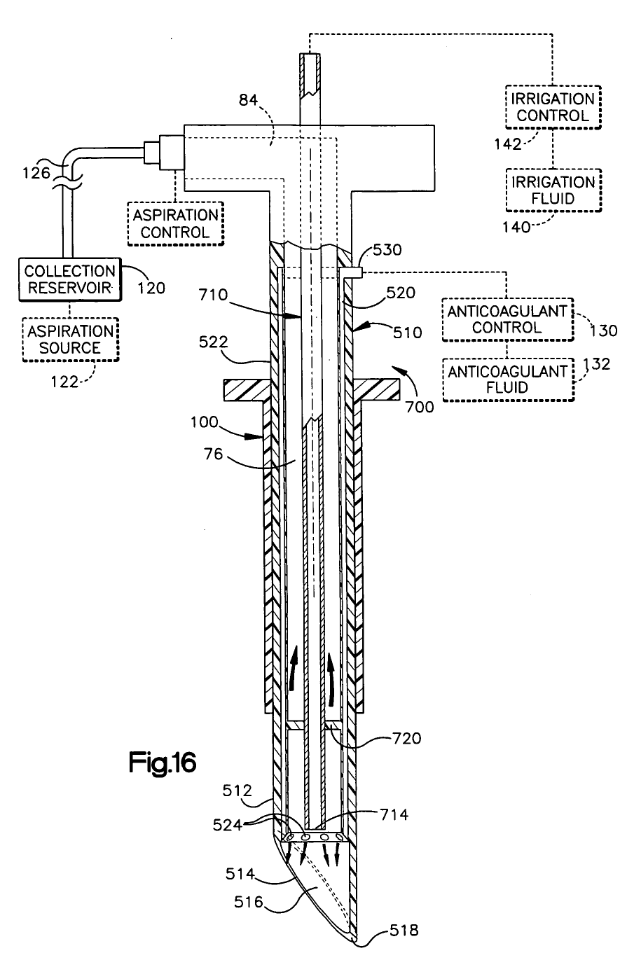

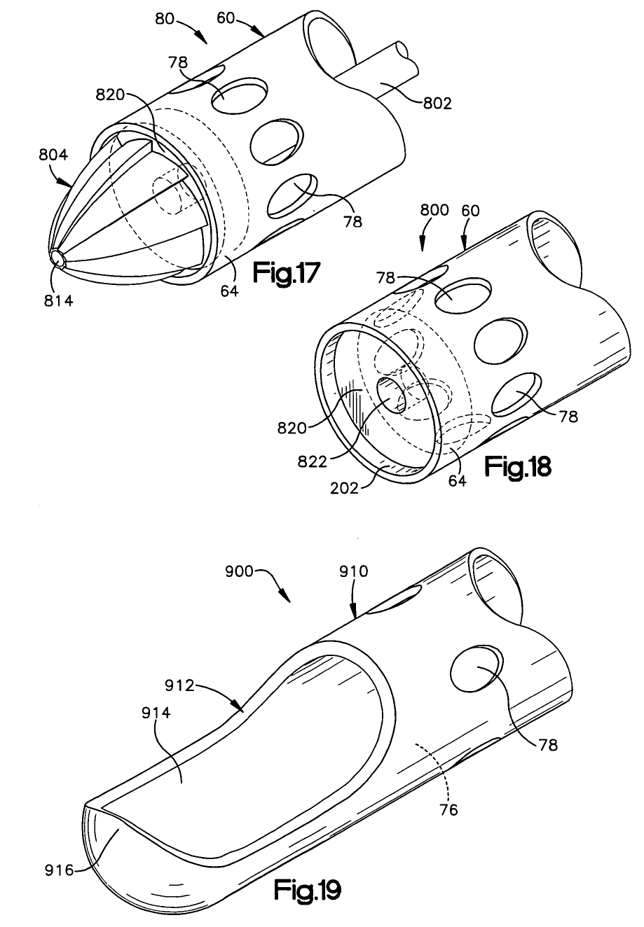

FIGS. 17 and 18 illustrate an apparatus 800 constructed in accordance with a seventh embodiment of the present invention. In FIGS. 17 and 18, reference numbers that are the same as reference numbers used in previously described embodiments identify parts and features that are the same as those in the previously described embodiments.

According to the seventh embodiment, the apparatus 800 includes a shaft 802 and a cutting bit 804. As best seen in FIG. 18, the distal end 64 of the cannula 60 includes a radially extending end wall 820 that closes a portion of the passage 76. The end wall 820 includes an axially extending passage 822 through which the shaft 802 projects.

The cutting bit 804 is similar to the cutting bits 210 and 210′ illustrated in FIGS. 6 and 6A, respectively. An aperture 814 is located at the distal tip of the cutting bit 804 to allow for irrigation.

The apparatus 800 is used to harvest bone marrow cells, blood, and bone fragments in much the same fashion as the apparatus described above, except that all of the harvested bone marrow cells, blood, and bone fragments are aspirated into the passage 76 in the cannula 60 through the radial apertures 78. This allows for harvesting large volumes of bone in a minimally invasive fashion yet, due to the relatively atraumatic nature of the apparatus 800, increases the number of harvested cells that are viable for bone marrow transplantation, bone grafting, and/or tissue engineering. Further, the morbidity of bone marrow harvest and the time required for marrow harvest is decreased.

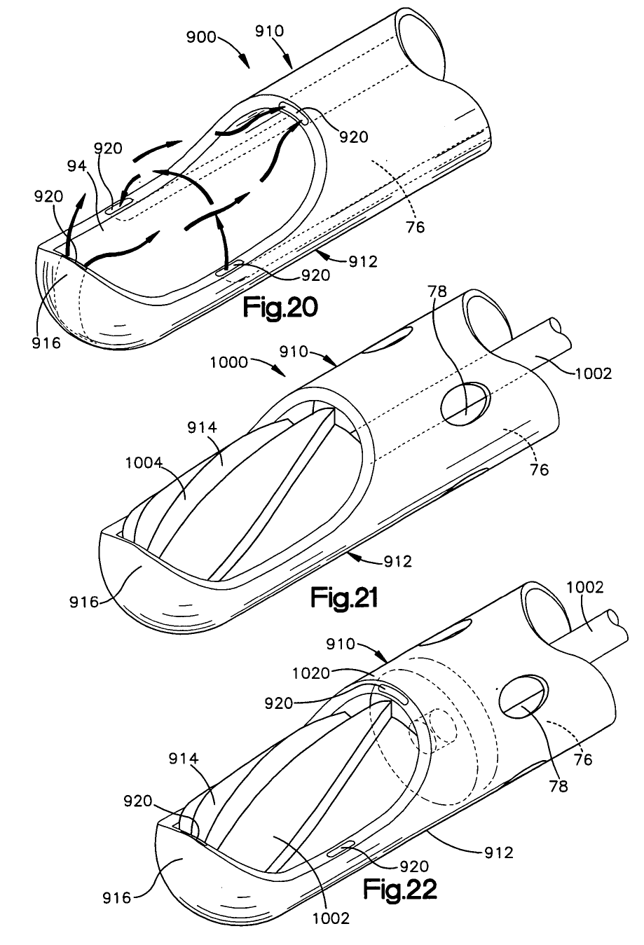

FIGS. 19 and 20 illustrate an apparatus 900 constructed in accordance with an eighth embodiment of the present invention. In FIGS. 19 and 20, reference numbers that are the same as reference numbers used in previously described embodiments identify parts and features that are the same as those in the previously described embodiments.

According to the eighth embodiment, the apparatus 900 includes a cannula 910 with a distal end 912 that includes a large, radially oriented opening 914. The radial opening 914 is open to the internal passage 76. A distal tip portion 916 of the cannula 910 is closed. The cannula 910 thus resembles a curette needle.

FIG. 20 illustrates an alternate embodiment of the cannula 910 in which a plurality of fluid nozzles 920 are located in the side wall of the cannula surrounding the opening 914. The nozzles 920 are fluidly connected by internal lumens (not shown) in the cannula 910 to the anticoagulant fluid source 130 and associated control 132.

The apparatus 900 of FIGS. 19 and 20 is used to harvest bone marrow cells, blood, and bone fragments in much the same manner as the apparatus 20 described above, except that the distal end 912 of the rigid cannula 910 and, in particular, the distal tip 916 are the structure that is used to disrupt, scrape, and excavate cancellous bone from the intramedullary canal. The distal end 912 of the cannula 910 is manually moved in both axial and radial directions to achieve this excavation. Irrigation fluid supplied to the distal 912 via the nozzles 920 can assist in washing the disrupted bone tissue into the opening 914 and into the passage 76 in the cannula 910.

As with the previous embodiments, the apparatus 900 and method allows for harvesting large volumes of bone in a minimally invasive fashion, increases the number of harvested cells that are available for bone marrow transplantation, bone grafting, and/or tissue engineering, while decreasing the morbidity of bone marrow harvest and the time required for marrow harvest.

FIG. 21 illustrates an apparatus 1000 constructed in accordance with a ninth embodiment of the present invention. In FIG. 21, reference numbers that are the same as reference numbers used in previously described embodiments identify parts and features that are the same as those in the previously described embodiments.

According to the ninth embodiment, the apparatus 1000 includes a shaft 1002 disposed with the cannula 910 and a cutting bit 1004 attached to the shaft. The cutting bit 1004 is disposed within the radial opening 916 in the distal end 912 of the cannula.

The apparatus 1000 is used to harvest bone marrow cells, blood, and bone fragments in much the same fashion as the apparatus described above, except that the cutting bit 1004 is semi-shielded within the distal end 912 of the cannula, which helps to reduce the trauma of the harvest and thereby increase the number of harvested cells that are viable for bone marrow transplantation, bone grafting, and/or tissue engineering.

FIG. 22 illustrates an apparatus 1100 constructed in accordance with a tenth embodiment of the present invention. In FIG. 22, reference numbers that are the same as reference numbers used in previously described embodiments identify parts and features that are the same as those in the previously described embodiments.

According to the tenth embodiment, the apparatus 1100 includes a radially extending end wall 1020 that closes a portion of the internal passage 76 in the cannula 910. The end wall 1020 includes an axially extending passage (not shown) through which the shaft 1002 projects.

The apparatus 1100 is used to harvest bone marrow cells, blood, and bone fragments in much the same fashion as the apparatus described above, except that all of the harvested bone marrow cells, blood, and bone fragments are aspirated into the passage 76 in the cannula 910 through the radial apertures 78. This allows for harvesting large volumes of bone in a minimally invasive fashion yet, due to the relatively atraumatic nature of the apparatus 1100, increases the number of harvested cells that are viable for bone marrow transplantation, bone grafting, and/or tissue engineering. Further, the apparatus 1100 of FIG. 21 allows the anticoagulant fluid to be introduced sooner into the disrupted bone tissue via the nozzles 920. The ability to introduce the anticoagulant fluid just after the harvested materials enter the passage 76 helps to preserve a high level of viability and to maintain the bone marrow cells harvested in suspension without formation of a fibrin clot.

From the above description of the invention, those skilled in the art will perceive improvements, changes and modifications. For example, it is contemplated that the present invention could be used adapted to harvest bone marrow cells, blood, and bone fragments from a variety of other bones including, but not limited to, the femur, the tibia, and the spine. Such improvements, changes and modifications within the skill of the art are intended to be covered by the appended claims.

Having described the invention, I claim:

1. A minimally invasive apparatus for harvesting bone marrow cells, blood, and bone fragments, said apparatus comprising:
a rigid cannula having a proximal end and a distal end with an opening, said distal end including a cutting tip that is movable axially and radially to cut and disrupt bone tissue while preserving necessary viability among harvested marrow cells, said cannula further including an inner surface defining an internal passage that extends from said opening toward said proximal end; and
means for applying suction to said internal passage in said cannula for drawing bone marrow cells, blood, and bone fragments disrupted from the bone tissue by said cutting tip into said internal passage for collection.

2. The apparatus of claim 1 further comprising control means for controlling said means for applying suction.

3. The apparatus of claim 1 further comprising means for controllably supplying irrigation fluid into said internal passage in said cannula.

4. The apparatus of claim 3 wherein said means for controllably supplying irrigation fluid is operatively coupled to control means for controlling said means for applying suction in order that irrigation fluid flow and suction can be oscillated so as not to coincide.

5. The apparatus of claim 1 further comprising means for controllably injecting an anticoagulant fluid into the harvested bone marrow cells, blood, and bone fragments during collection.

6. The apparatus of claim 5 wherein said cannula further includes a plurality of nozzles adjacent said distal end for introducing anticoagulant fluid into harvested bone marrow cells, blood, and bone fragments immediately following their harvest.

7. The apparatus of claim 1 further comprising a collection chamber in fluid communication with said internal passage for receiving and collecting the harvested bone marrow cells, blood, and bone fragments.

8. The apparatus of claim 1 further comprising a sheath disposed co-axially about a portion of said cannula for providing and maintaining a single percutaneous puncture site.

9. The apparatus of claim 1 further comprising a rotatable shaft disposed co-axially within said internal passage in said cannula, said shaft having a distal end with a cutting bit for cutting and disrupting bone tissue while preserving necessary viability among harvested marrow cells, said cutting bit projecting through said opening in said cannula.

10. The apparatus of claim 9 further comprising means for rotating said shaft and said cutting bit.

11. The apparatus of claim 10 further comprising control means for controlling said means for rotating said shaft.

12. The apparatus of claim 9 wherein said cannula includes a radially extending end wall that closes a portion of said opening at said distal end of said cannula, said end wall having an axially extending passage through which said shaft projects.

13. The apparatus of claim 12 wherein said cannula includes a plurality of circumferentially spaced radially extending apertures through which bone marrow cells, blood, and bone fragments disrupted from the bone tissue are aspirated by said means for applying suction.

14. The apparatus of claim 1 wherein said cannula includes a plurality of circumferentially spaced radially extending apertures through which bone marrow cells, blood, and bone fragments disrupted from the bone tissue are aspirated by said means for applying suction.

15. The apparatus of claim 1 wherein said opening at said distal end of said cannula extends in the radial direction through said inner surface of said cannula.

16. The apparatus of claim 15 wherein said distal end of said cannula includes a plurality of third nozzles adjacent said radial opening for introducing an anticoagulant fluid into harvested bone marrow cells, blood, and bone fragments.

17. The apparatus of claim 15 further comprising a rotatable shaft disposed coaxially with said internal passage in said cannula, said shaft having a distal end with a cutting bit for cutting and disrupting bone tissue while preserving necessary viability among harvested marrow cells, said cutting bit being disposed within said radial opening in said cannula.

18. The apparatus of claim 17 wherein said cannula includes a radially extending wall that closes off a portion of said internal passage at said distal end of said cannula, said end wall having an axially extending passage through which said shaft projects.

19. The apparatus of claim 18 wherein said cannula includes a plurality of circumferentially spaced radially extending apertures through which bone marrow cells, blood, and bone fragments disrupted from the bone tissue are aspirated by said means for applying suction.

20. A minimally invasive apparatus for harvesting bone marrow cells, blood, and bone fragments, said apparatus comprising:
a rotatable shaft having a distal end, said distal end including a cutting bit for cutting and disrupting bone tissue while preserving necessary viability among harvested marrow cells;
means for rotating said shaft;
a rigid cannula disposed co-axially about at least a portion of said shaft, said cannula having a distal end with an opening through which said cutting bit extends, said cannula further having an inner surface defining an internal passage that extends from said opening toward a proximal end of said cannula; and
means for applying suction to said internal passage in said cannula for drawing bone marrow cells, blood, and bone fragments disrupted from the bone tissue by said cutting bit into said internal passage for collection.

21. The apparatus of claim 20 further comprising control means for controlling said means for rotating said shaft.

22. The apparatus of claim 20 further comprising control means for controlling said means for applying suction.

23. The apparatus of claim 20 further comprising means for controllably supplying irrigation fluid to said distal end of said shaft.

24. The apparatus of claim 20 further comprising means for controllably supplying irrigation fluid into said internal passage in said cannula.

25. The apparatus of claim 24 wherein said means for controllably supplying irrigation fluid is operatively coupled to control means for controlling said means for applying suction in order that irrigation fluid flow and suction can be oscillated so as not to coincide.

26. The apparatus of claim 20 further comprising means for controllably injecting an anticoagulant fluid into the harvested bone marrow cells, blood, and bone fragments during collection.

27. The apparatus of claim 26 wherein said cannula further includes a plurality of nozzles adjacent said distal end for introducing anticoagulant fluid into harvested bone marrow cells, blood, and bone fragments immediately following their harvest.

28. The apparatus of claim 20 further comprising a collection chamber in fluid communication with said internal passage for receiving and collecting the harvested bone marrow cells, blood, and bone fragments.

29. The apparatus of claim 20 further comprising a sheath disposed coaxially about a portion of said cannula for providing and maintaining a single percutaneous puncture site.

30. The apparatus of claim 20 wherein said cannula includes a plurality of circumferentially spaced radially extending apertures through which bone marrow cells, blood, and bone fragments disrupted from the bone tissue are aspirated by said means for applying suction.

31. The apparatus of claim 30 wherein said cannula includes a radially extending end wall that closes a portion of said opening at said distal end of said cannula, said end wall having an axially extending passage through which said shaft projects.

32. The apparatus of claim 20 wherein said distal end of said cannula includes a cutting tip that is movable axially and radially to cut and disrupt bone tissue while preserving necessary viability among harvested marrow cells.

33. The apparatus of claim 20 wherein said opening at said distal end of said cannula extends in the radial direction through said inner surface of said cannula.

34. The apparatus of claim 16 wherein said distal end of said cannula includes a plurality of third nozzles adjacent said radial opening for introducing an anticoagulant fluid into harvested bone marrow cells, blood, and bone fragments.

35. The apparatus of claim 32 wherein said cannula includes a radially extending wall that closes off a portion of said internal passage at said distal end of said cannula, said end wall having an axially extending passage through which said shaft projects.

36. The apparatus of claim 34 wherein said cannula includes a plurality of circumferentially spaced radially extending apertures through which bone marrow cells, blood, and bone fragments disrupted from the bone tissue are aspirated by said means for applying suction.

37. A minimally invasive method for harvesting bone marrow cells, blood, and bone fragments, said method comprising the steps of:
(a) providing a cannula having a proximal end and a distal end with an opening, the distal end including a cutting tip for disrupting bone tissue while preserving necessary viability among harvested marrow cells, the cannula further including an inner surface defining an internal passage that extends from the opening toward the proximal end;
(b) inserting the distal end of the cannula through a puncture site, through the cortex of a bone, and into the intramedullary canal of the bone;
(c) applying suction to the internal passage which draws bone marrow cells, blood, and bone fragments disrupted from the cancellous bone into the internal passage for collection;
(d) manually moving the distal end of the cannula in both axial and radial directions within the intramedullary canal to cut and disrupt the bone tissue;
(e) manually moving the distal end of the cannula to different locations in the cancellous bone and disrupting additional bone tissue with the apparatus remaining in the same puncture site; and
(f) repeating steps (d) and (e) to further collect bone marrow cells, blood, and bone fragments.

38. The method of claim 37 further comprising the step of supplying irrigation fluid to the distal end of the cannula to minimize thermal or mechanical trauma to the harvested cells and to help carry the harvested bone marrow cells, blood, and bone fragments into the passage.

39. The method of claim 37 further comprising the step of supplying an anticoagulant fluid into the harvested bone marrow cells, blood, and bone fragments to inhibit clot formation.

40. The method of claim 39 further comprising the step of oscillating the supply of irrigation fluid and the supply of anticoagulant fluid.

41. The method of claim 39 further comprising the step of supplying the anticoagulant fluid at a location adjacent the distal end of the cannula.

42. The method of claim 37 further comprising the steps of:
providing a collection reservoir for collecting the harvested bone marrow cells, blood, and bone fragments; and
fluidly connecting the internal passage in the cannula with the collection reservoir.

43. The method of claim 37 further comprising the steps of:
providing a sheath that is disposed co-axially about a portion of the cannula;
inserting the sheath percutaneously to the cortex of the bone so that the sheath provides and maintains a single percutaneous puncture site for harvesting bone marrow cells, blood, and bone fragments.

44. The method of claim 37 further comprising the steps of:
providing a rotatable shaft disposed co-axially within the internal passage in the cannula, the shaft having distal end with a cutting bit that projects through the opening in the cannula; and
rotating the shaft and the cutting bit to cut and disrupt bone tissue in the intramedullary canal while preserving necessary viability among harvested marrow cells.

45. The method of claim 37 further comprising the step of aspirating bone marrow cells, blood, and bone fragments disrupted from the bone tissue through a plurality of circumferentially spaced radially oriented apertures at the distal end of the cannula.

46. A minimally invasive method for harvesting bone marrow cells, blood, and bone fragments, said method comprising the steps of:
(a) providing an apparatus having a rotatable shaft with a distal end for disrupting bone tissue, the apparatus further including means for rotating the shaft and a cannula encircling the shaft to define an annular passage;
(b) inserting the distal end of the shaft through a puncture site, through the cortex of a bone, and into the intramedullary canal of the bone;
(c) rotating the shaft to cause the cutting bit to rotate and disrupt the cancellous bone in the intramedullary canal;
(d) applying suction to the annular passage which draws bone marrow cells, blood, and bone fragments disrupted from the cancellous bone into the annular passage for collection;
(e) manually moving the distal end of the shaft to different locations in the cancellous bone and disrupting additional bone tissue with the apparatus remaining in the same puncture site; and
(f) repeating steps (c) and (d) to further collect bone marrow cells, blood, and bone fragments.

47. The method of claim 46 further comprising the step of supplying irrigation fluid to the distal end of the shaft to minimize thermal or mechanical trauma to the harvested cells and to help carry the harvested bone marrow cells, blood, and bone fragments into the passage.

48. The method of claim 46 further comprising the step of supplying an anticoagulant fluid into the harvested bone marrow cells, blood, and bone fragments to inhibit clot formation.

49. The method of claim 48 further comprising the step of oscillating the supply of irrigation fluid and the supply of anticoagulant fluid.

50. The method of claim 48 further comprising the step of supplying the anticoagulant fluid at a location adjacent the distal end of the cannula.

51. The method of claim 46 further comprising the steps of:
providing a collection reservoir for collecting the harvested bone marrow cells, blood, and bone fragments; and
fluidly connecting the passage in the cannula with the collection reservoir.

52. The method of claim 46 further comprising the steps of:
providing a sheath that is disposed co-axially about a portion of the cannula;
inserting the sheath percutaneously to the cortex of the bone so that the sheath provides and maintains a single percutaneous puncture site for harvesting bone marrow cells, blood, and bone fragments.

53. The method of claim 46 further comprising the steps of:
(a) manually moving the distal end of the cannula in both axial and radial directions within the intramedullary canal to cut and disrupt the bone tissue;
(b) manually moving the distal end of the cannula to different locations in the cancellous bone and disrupting additional bone tissue with the apparatus remaining in the same puncture site; and
(c) repeating steps (d) and (e) to further collect bone marrow cells, blood, and bone fragments.

54. The apparatus of claim 46 further comprising the step of aspirating bone marrow cells, blood, and bone fragments disrupted from the bone tissue through a plurality of circumferentially spaced radially oriented apertures at the distal end of the cannula.
